(19) United States
(12) Patent Application Publication
(10) Pub. No.: US 2008/0051860 A1
(43) Pub. Date: Feb. 28, 2008

(54) Cosmetic mask and method of using

(76) Inventor: Johan David Van Wyk, Randburg (ZA)

Correspondence Address: JAMES RAY & ASSOCIATES, 2640 Pitcairn Road, Monroeville, PA, 15146, US

(21) Appl. No.: 11/894,901

(22) Filed: Aug. 22, 2007

(30) Foreign Application Priority Data
Aug. 22, 2006 (ZA) 2006/07007

(51) Int. Cl. A61F 7/00; A45D 24/00; A61H 33/04; A61M 35/00; A45D 40/30

(52) U.S. Cl. 607/109; 132/200; 132/319; 604/289; 604/303

(57) ABSTRACT

A multi-layer cosmetic mask designed to enhance the absorption of skin care compositions is disclosed. The mask comprises a thermally reflective layer, composed of foil or similar material, and a thermally insulating layer, composed of fleece or similar material. A method of using the cosmetic mask is also disclosed, comprising the steps of applying a moisturizing composition to the skin, applying the thermally reflective foil layer over the moisturizer, and then applying the thermally insulating mask layer over the foil layer. The combination of layers increases the temperature under the mask, and thereby increases the absorption of the moisturizing composition into the skin of a user.

### CROSS REFERENCE TO RELATED APPLICATION

This patent application is related to and claims priority from South African Provisional Patent Application Serial Number 2006/07007, filed Aug. 22, 2006.

### FIELD OF THE INVENTION

The present invention relates to a cosmetic mask, and a method of using a cosmetic mask to enhance the effectiveness of skin care products.

### BACKGROUND OF THE INVENTION

Many topically applied skin care products, both cosmetic and pharmaceutical, are known in industry, and are widely used for a variety of purposes. The efficiency and efficacy of such skin care products is directly proportional to the efficiency of absorption through the skin. Percutaneous absorption is a complex problem, as the skin is relatively impermeable. The barrier to permeability is situated in the stratum corneum, or horny layer, of the epidermis. Increased hydration of the stratum corneum reduces its barrier function, facilitating the absorption of a variety of molecules.

Many methods have been proposed in the past to enhance percutaneous absorption, including ultrasound, iontophoresis, variation of the physico/chemical properties of the penetrants, and use of specific carriers to carry active ingredients into the skin. These methods are often complicated, and can require advanced technical equipment or procedures to exercise. There exists a need in the art for a simple, inexpensive method of enhancing percutaneous absorption, and the cosmetic mask of the present invention has been developed to meet this need.

There are numerous examples of cosmetic masks in the prior art. U.S. Pat. No. 4,886,079 to Mooney discloses a cosmetic template which is heated and pressed against the face. The mask disclosed by Mooney includes a plurality of small circles, and is used as a stencil template to assist in the application of cosmetics. The form-fitting mask disclosed by Mooney is not intended to enhance the absorption of skin care compositions.

U.S. Pat. No. 5,660,840 to Pruett discloses a therapeutic cosmetic composition and method of applying the composition using a pliable plastic facial mask. Pruett does not disclose a mask specifically designed to enhance absorption.

U.S. Pat. No. 5,607,408 to Boucly discloses a flexible opaque mask useful in the application of skin treatment solutions. The mask includes a plurality of openings connected to tubes, through which treatment solutions are dispensed. The mask disclosed by Boucly is preferably constructed of flexible opaque plastic.

U.S. Pat. No. 5,706,828 to Shiota discloses a sudorific mask, designed to increase perspiration on the facial skin in a comfortable manner. No method to use the mask to enhance the action of skin care compositions is disclosed. The mask disclosed by Shiota is constructed of a single layer of plastic material, such as polyethylene.

The cosmetic mask of the present invention was designed to overcome the disadvantages of the cosmetic masks in the prior art, including the examples detailed above. The invention is a simple multi-layer mask constructed from materials specifically chosen to increase the temperature of the skin surface and enhance absorption of skin care compositions. The mask combines the effects of occlusion, i.e. the covering of the skin with air-tight material to increase the hydration of the skin, and reflective thermal radiation, which increases the temperature at the surface of the skin. The combination of occlusion and reflective thermal radiation diminishes the effectiveness of the barrier function of the skin, increasing the permeation of the skin by moisturizing or other skin care compositions.

### SUMMARY OF THE INVENTION

The present invention provides a cosmetic mask designed to enhance the absorption of skin care compositions by the skin of the user. The mask comprises an inner layer of thermally reflective material, such as metal foil, an outer layer of thermally insulating material, such as fleece, and means to secure the mask on the face of a user, such as ear loops, bands, straps, hook and loop type fasteners, and the like. The inner and outer layers of the mask are shaped to the contours of the skin surface. In a preferred embodiment, the skin surface would be that of the face, but the mask can be adapted for use on other parts of the body. When used as a facial mask, areas are cut out for the eyes, mouth and nose of the user. The fastening means are preferably attached to the outer layer of the mask.

In one embodiment of the invention, an additional layer of moisture-impermeable material, such as polyethylene film, is disposed between the inner layer and the outer layer. In another embodiment, the moisture impermeable layer is disposed between the inner layer and the shin of the user.

In one embodiment of the invention, the outer layer of the mask is comprised of two sub-layers, including space between the sub-layers to position an external source of heat, such as an electric heating pad or similar device.

The present invention further includes a method of using the cosmetic mask disclosed above, comprising the steps of application of a skin care composition on the skin of the user, applying a first layer of thermally reflective material over the coating of cosmetic composition, securing a layer of thermally insulating material over the first layer, and fastening the mask on the user for a time period sufficient for the absorption of the cosmetic composition. The inner layer of the mask as used in this method would preferably be disposable, while the outer layer of the mask would be washable and reusable.

### OBJECTS OF THE INVENTION

It is, therefore, one of the primary objects of the present invention to provide a cosmetic mask designed to enhance the absorption of skin care compositions.

Another object of the present invention is to provide a simple method to apply skin care compositions with enhanced effectiveness.

In addition to the various objects and advantages of the present invention described with some degree of specificity above it should be obvious that additional objects and advantages of the present invention will become more readily apparent to those persons who are skilled in the relevant art from the following more detailed description of the invention, particularly, when such description is taken in conjunction with the attached drawing figures and with the appended claims.

### BRIEF DESCRIPTION OF THE DRAWINGS

FIG. 1 is a cross-sectional side view of the cosmetic mask of the present invention, as applied to the skin of a user.

FIG. 2 is a cross-sectional side view of a second embodiment of the invention, as applied to the skin of a user.

FIG. 3 is a perspective view of the cosmetic mask as worn by a user.

### BRIEF DESCRIPTION OF A PRESENTLY PREFERRED AND VARIOUS ALTERNATIVE EMBODIMENTS OF THE INVENTION

Prior to proceeding to the more detailed description of the present invention it should be noted that, for the sake of clarity and understanding, identical components which have identical functions have been identified with identical reference numerals throughout the several views illustrated in the drawing figures.

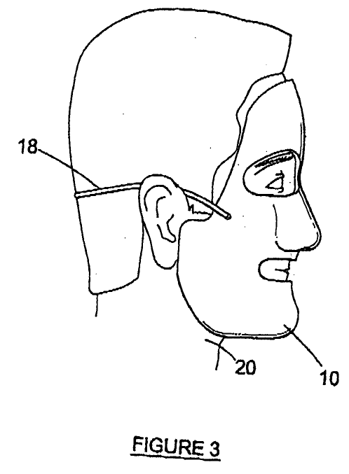

Reference is now made, more particularly, to FIG. 3, which illustrates the cosmetic mask 10 of the invention in place on the skin 20 of a user. Fastening means 18 hold the mask on the face of the user. Areas are cut out of the mask for the eyes, nostrils and mouth of the user.

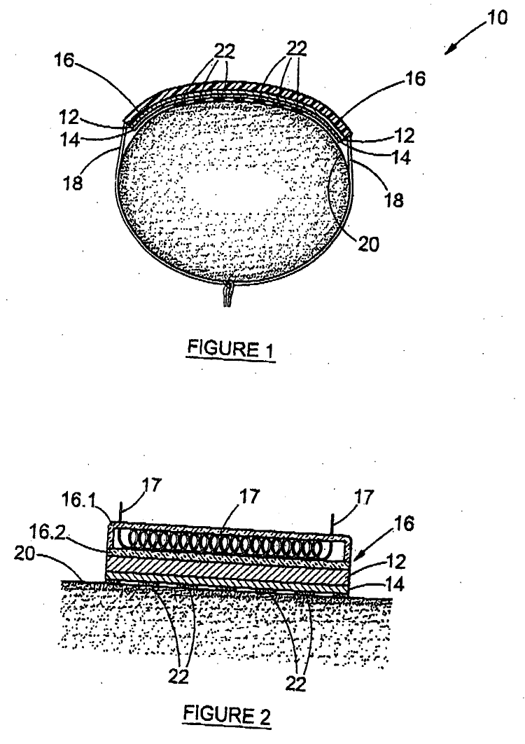

FIG. 1 illustrates the design and operation of the mask in greater detail. The mask 10 is composed of at least two layers. An inner layer 12 is made from a material having at least one reflective surface, such as aluminum or gold foil, which enables body heat to be reflected back to the surface of the skin 20 of the user. An outer layer 16 is made from a thermally insulating material such as fleece. Other suitable materials, including cloth, polyester resins, plastic, rubber, paper, or stretchable fabrics such as LYCRA™ could be selected by one of skill in the art. Outer layer 16 provides structural support and insulation to the mask, and may be shaped to form the face of a user. FIG. 1 illustrates an optional third layer 14, constructed of impermeable material and positioned between inner layer 12 and outer layer 16. The impermeable material is preferably polyethylene film or similar materials known in the art, and serves as an additional insulating layer. This optional layer 14 may be positioned between the inner layer 12 and outer layer 16, or may be positioned between inner layer 12 and the skin 20 of the user.

As illustrated in FIG. 1, a suitable amount of cosmetic product 22 is applied to the skin 20 of the user. The product 22 can be a moisturizing, rejuvenating, anti-aging, anti-pigmentation, cosmetic or pharmaceutical cream. Inner layer 12 is then applied over the coating of cosmetic product 22, and if desired, third layer 14 may be applied over inner layer 12. The third layer 14 may also be applied directly over the cosmetic composition, with inner layer 12 then applied over layer 14. The outer layer 16, with attached fastening means 18, is applied last. The fastening means 18 illustrated in FIG. 1 are strings which are tied behind the head of the user, but other fastening means such as elastic bands or hook and loop type fasteners could be employed. In a preferred embodiment, the mask is attached to the face by a pair of ear loops positioned on either side of the mask.

Note that the configuration of the inner, outer, and third layers may be selected by the user. The third layer 14 may be omitted altogether, but the rise in skin surface temperature achieved by the mask is higher when layer 14 is included. Inner layer 12, made of thermally reflective material, must be positioned such that a reflective surface of the layer faces the skin of the user, ensuring that heat emitted by the body is reflected back toward the skin.

An additional embodiment of the invention is illustrated in FIG. 2. In this embodiment, outer layer 16 is comprised of two sub-layers, 16.1 and 16.2, and an externally heatable heat source 17 is positioned between the two layers. Heat source 17 is preferably an electric heating element such as a heating pad.

While a presently preferred and various alternative embodiments of the present invention have been described in sufficient detail above to enable a person skilled in the relevant art to make and use the same it should be obvious that various other adaptations and modifications can be envisioned by those persons skilled in such art without departing from either the spirit of the invention or the scope of the appended claims.

I claim:

1. A cosmetic mask for increasing the absorption of skin moisturizing compositions, comprising
a) an inner layer of thermally reflective material in contact with a skin surface;
b) at least one outer layer of thermally insulating material, where said inner layer and said outer layer are shaped to the contours of said skin surface; and
c) means for securing said mask to said skin surface.

2. A cosmetic mask as recited in claim 1, wherein said skin surface is facial skin.

3. A cosmetic mask as recited in claim 2, further including cutout areas to accommodate the eyes, nose and mouth of a user.

4. A cosmetic mask as recited in claim 3, wherein means for securing said mask to the face are selected from the group of ear loops, elastic bands, and hook and loop fasteners.

5. A cosmetic mask as recited in claim 1, wherein said inner layer is a foil layer.

6. A cosmetic mask as recited in claim 5, wherein said outer layer is selected from the group of fleece, cloth, plastic, paper, and rubber.

7. A cosmetic mask as recited in claim 1, further including a layer of moisture-impermeable material positioned between said thermally reflecting inner layer and said thermally insulating outer layer.

8. A cosmetic mask as recited in claim 7, where said layer of moisture-impermeable material is polyethylene film.

9. A cosmetic mask as recited in claim 1, further including a layer of moisture-impermeable material positioned between said thermally reflecting inner layer and said skin surface.

10. A cosmetic mask as recited in claim 9, where said layer of moisture-impermeable material is polyethylene film.

11. A cosmetic mask as recited in claim 1, wherein said at least one outer layer comprises two sub-layers, with a source of heat sandwiched between said two sub-layers.

12. A cosmetic mask as recited in claim 11, wherein said source of heat is an electric heating element.

13. A method for enhancing percutaneous absorption of cosmetic compositions comprising:
a) applying a coating of said cosmetic composition to a skin surface;
b) adhering a layer of thermally reflective material to said coating of said cosmetic composition;
c) securing a layer of thermally insulating material over said layer of thermally reflective material; and
d) providing a sufficient time period for the absorption of said cosmetic composition by said skin surface.
